(19) United States
(12) Patent Application Publication
(10) Pub. No.: US 2024/0382328 A1
(43) Pub. Date: Nov. 21, 2024

(54) ANCILLARY DEVICE AND KIT FOR ANASTOMOSIS

(71) Applicant: EASY VASCULAR CURE, CRETEIL (FR)

(72) Inventor: Salam ABOU TAAM, TOULOUSE (FR)

(21) Appl. No.: 18/693,721

(22) PCT Filed: Sep. 21, 2022
(86) PCT No.: PCT/EP2022/076238

§ 371 Date: Mar. 20, 2024

(30) Foreign Application Priority Data
Sep. 21, 2021 (EP) 21198058.6

(51) Int. Cl. A61F 2/958; A61B 17/11; A61B 90/00

(52) U.S. Cl. CPC A61F 2/958; A61B 17/11; A61B 90/06; A61B 2017/1107; A61B 2090/061; A61F 2002/9583

(57) ABSTRACT

An ancillary device capable of assisting in the two anastomosis procedures necessary for replacing a segment of a corporeal duct with a prosthesis, and the connection of said prosthesis without the need for any suture. An optimised kit for anastomosis including the ancillary device is also provided. The ancillary device for anastomosis, including an elongate body provided with a longitudinal through channel intended to receive a tubular prosthesis therein, the body of the device including, on a proximal side, a handling portion and, on a distal side, a shaping assembly formed by a plurality of the lamellae defining a compression area of the prosthesis. The body of the device includes a closable longitudinal opening giving access to the longitudinal channel intended to receive the prosthesis.

### CROSS REFERENCE TO RELATED APPLICATIONS

This application is a National Stage of International Application No. PCT/EP2022/076238, having an International Filing Date of 21 Sep. 2022, which designated the United States of America, and which International Application was published under PCT Article 21 (2) as WO Publication No. 2023/046759 A1, which claims priority from and the benefit of European Patent Application No. 21198058.6 filed on 21 Sep. 2021, the disclosures of which are incorporated herein by reference in their entireties.

### BACKGROUND

### Field

The present disclosure relates to the field of devices for assisting in anastomosis interventions. More particularly, the disclosure relates to an ancillary device for placing a connection assembly in a corporeal duct for making a suture between two extracorporeal and corporeal ducts. The disclosure also relates to a kit including said ancillary device and at least one connection assembly including a radial expansion sleeve with spikes and an inflation balloon.

### Brief Description of Related Developments

In the present disclosure, an anastomosis refers to a connection between two tubular corporeal structures, such as blood vessels, bronchi or intestine segments. Anastomoses are very common to remedy vascular problems, for example to treat a thrombosed or aneurysm artery by sectioning and removing its pathological portion, and then reconnecting the two segments of the artery by means of a prosthesis.

To reconnect vessel segments, it is known to carry out a surgical manual suture. This type of anastomosis, which is the most frequent in vascular surgery, allows addressing most arterial repair clinical situations. However, it is observed that the surgery time required to carry out such a manual suture is relatively long. Yet, during a vascular repair, for example, the anastomosis requires a vascular clamping to avoid any haemorrhage. The duration of this clamping, on the one hand, is a proof of cardiac force for the patient that could lead to a coronary syndrome and, on the other hand, generates ischemia of the vascularised tissues by the downstream arterial axis.

Furthermore, such a manual procedure imposes a wide accessibility for the practitioner to be able to carry out the operating gestures necessary for the connection of the two segments to one another. This accessibility is as determining as the dexterity of the practitioner in the proper achievement of the suture.

Nonetheless, such an accessibility can be obtained only throughout a large exposed operating surface, which leads, for example, in the context of aortic surgery, to negative impacts on post-operative pains, resumption of intestinal transit, pneumopathies, coronaropathies and hospital stay durations. Conventional mini-approach surgery, as well as laparoscopic aortic surgery, allow solving such drawbacks and reducing the aforementioned effects on the patient.

Nevertheless, conventional mini-approach surgery laparoscopic aortic surgery offers reduced accessibility conditions, making it difficult to carry out the surgical gestures necessary for vascular suturing by removing a section of the vessel and connecting the resulting two ends using a prosthesis.

In order to substitute this manual suture, it is known to use a stent (or connector) that is very particular in terms of design and function in the form of an expandable metal sleeve having spikes at its external surface. This stent with spikes is mounted on an inflation balloon and inserted at one end of the prosthesis to be connected with the corporeal duct. When the end of the prosthesis is inserted into the duct, the balloon is inflated so as to increase the diameter of the sleeve, the spikes thus perforate the prosthesis and the corporeal duct to ensure connection between these two elements.

This connection technique is very advantageous but poses a few problems in practice, in particular for positioning the stent with spikes in the prosthesis and the corporeal duct. Patent EP 2638 869 B1 proposes an ancillary device designed especially to facilitate an anastomosis using a stent with spikes and an inflation balloon. In particular, this document proposes an ancillary device including a hollow tubular body including a handling portion and a shaping assembly, said body including a through hole into which said prosthesis is inserted with the stent with spikes mounted on the balloon. The shaping assembly allows compressing the prosthesis sufficiently to enable insertion thereof into the corporeal duct, while limiting said compression so as not to damage the spikes or the prosthesis before the inflation step. This document also suggests using a protective enclosure surrounding the stent with spikes. Thus, the stent and the prosthesis are protected during handling thereof until insertion thereof into the corporeal duct. Of course, the enclosure should be removed before the inflation step.

In practice, said ancillary device is very advantageous for assisting in the placement of the prosthesis and of the stent with spikes during the first anastomosis. Nonetheless, this device is unsuitable for assisting in the second anastomosis. This means that, when a segment of a corporeal duct is replaced by a prosthesis, the complete replacement procedure requires two connections, a first anastomosis for connecting one end of the prosthesis to the so-called “high” end of the corporeal duct, and a second anastomosis for connecting the so-called “low” end of the corporeal duct and the other end of the prosthesis. Hence, the drawback of said ancillary device of the prior art consists in not being able to assist in the second anastomosis.

Indeed, a segment to be replaced of the corporeal duct, and therefore of the prosthesis, often has a limited length sometimes in the range of two centimetres. Once the first anastomosis is complete, the length of the free segment of the prosthesis is very limited. On the other hand, the body of the ancillary device has a length of about 20 cm, and the prosthesis is inserted into said device through an inlet opposite to the compression area of the prosthesis and from which said prosthesis is inserted into the duct. Hence, it is not possible to insert the second end of the prosthesis along the entire ancillary device, and up to said compression area. In addition, some difficulties are observed to properly position the balloon with the stent in the ancillary device, and to pull off the protective enclosure from the balloon.

### SUMMARY

An object of the present disclosure to propose an ancillary device capable of assisting in the two anastomosis procedures necessary to replace a segment of a corporeal duct by a prosthesis, and connecting said prosthesis without the need for any suture. The disclosure also relates to an optimised kit for anastomosis including said ancillary device.

To this end, the disclosure proposes an ancillary device for anastomosis, said device including an elongate body provided with a longitudinal through channel intended to receive a tubular prosthesis therein, the body of the device including, on a proximal side, a handling portion formed by a hollow enclosure, preferably tubular shaped and, on a distal side, a shaping assembly formed by a plurality of the lamellae defining a compression area of said prosthesis, characterised in that:
the body of the device includes a closable longitudinal opening giving access to the longitudinal channel at said handling portion and of the shaping assembly.

Advantageously, the longitudinal opening allows giving access to the channel of the device, and therefore accurately positioning the prosthesis at the shaping assembly, in particular when the length of the prosthesis is insufficient to handle said prosthesis after the first anastomosis. In addition, this closable opening allows easily disengaging the ancillary device following the insertion of the second end of the prosthesis into the corporeal duct, during the second anastomosis.

In a non-limiting aspect, the ancillary device may also include the following technical features in any technically-feasible combination:
the longitudinal opening is configured as an articulated opening splitting said body into two portions, and preferably into two symmetrical portions;
the longitudinal opening is configured as an opening splitting said body into two portions, and wherein the two portions are assembled together by means of at least one rail and a groove cooperating with one another and being respectively provided on each of the two portions along the body of the handling portion;
the device includes an open configuration, and a closed configuration respectively defined by opening or closure of said longitudinal opening, and a means for locking said closed configuration;
the device includes an open configuration, and a closed configuration controllable by the engagement and disengagement of the rail in the groove, and preferably a means for locking said closed configuration;
the lamellae of the shaping assembly are arranged circumferentially and according to predefined intervals around the handling portion and according to an axis of the device, and in which device the lamellae include a released position and a clamped position defining respectively an initial diameter and a reduced diameter of the channel receiving the prosthesis at said compression area;
the handling portion includes one or more projecting portion(s) at a junction area with the lamellae of the shaping assembly, said projecting portion allowing radially compressing the lamellae when it is pushed towards the inside of the device;
the device includes an external ring movable in translation along the body of the device, said ring being able to fit onto the projecting portion(s) to activate the closed position of lamellae. This movable external ring also allows securing the two symmetrical portions of the body in the closed position;
said device includes an actuating means configured to radially compress said lamellae and activate their clamped position, such as said external ring;

The disclosure also relates to a kit for anastomosis optimised to cooperate with the ancillary device of the disclosure, said kit including:
an ancillary device according to one of the preceding claims;
a connection assembly including at least one radial expansion sleeve with spikes and an inflation balloon; and preferably
a vascular prosthesis or substitute.

In one aspect, the kit includes an insertion guide system including a set of stop elements on the body of the ancillary device and on the connection assembly, configured and arranged so as to limit the depth of insertion of the connection assembly into the channel of the ancillary device.

In one aspect, the inflation balloon includes a catheter for connecting said balloon to an inflation syringe, and said catheter includes one or more fin(s) configured to anchor to the body of the ancillary device or to the prosthesis, and limiting a depth of insertion of said inflation balloon into the channel of the ancillary device. Preferably, the fins include peripheral hooks configured to hook at a proximal end of the handling portion of the ancillary device. Preferably, the fins are movable in translation along the catheter and include a locked configuration and an unlocked configuration, respectively limiting or enabling mobility of the fins.

In one aspect, the kit includes a peelable or frangible protective enclosure surrounding the radial expansion sleeve with spikes. To this end, the enclosure includes at least one peelable or frangible longitudinal axis.

Finally, the kit also includes a diameter tester including at least one tubular body with a predefined diameter connected to a grip, and preferably a plurality of tubular bodies connected respectively to a grip. Said tubular bodies being intended to be inserted into a corporeal duct in order to determine a diameter of a prosthesis to be inserted, said diameter being identified when one of said tubular bodies is inserted into the corporeal duct with a limited clearance.

### BRIEF DESCRIPTION OF THE FIGURES

The disclosure will be better understood upon reading the following description and examining the appended figures. These are presented solely for indicative and non-limiting disclosure of the disclosure.

FIG. 1 An example of an anastomosis kit including an ancillary device according to the disclosure.

FIG. 2A is a schematic illustration of the released position and FIG. 2B is a schematic illustration of the clamped position of the shaping assembly of the ancillary device of the disclosure.

FIG. 3A schematic illustration of the removal of the ancillary device after the first anastomosis.

FIG. 4A schematic illustration of the ancillary device of the disclosure in the open configuration and of the reception of the prosthesis after the first anastomosis.

FIG. 5A schematic illustration of the ancillary device of the disclosure in the open configuration and of a variant of reception of the prosthesis after the first anastomosis.

FIG. 6A schematic illustration of the radial expansion of a stent with spikes in a corporeal duct.

FIG. 7A schematic illustration of the inflation balloon provided with a stop element of the anastomosis kit according to the disclosure.

FIG. 8A schematic illustration of the kit of the disclosure showing the protective enclosure inserted into the channel of the ancillary device.

FIG. 9A schematic illustration of a diameter tester of the kit of the disclosure.

FIG. 10A schematic illustration of a cross-section of the handling portion according to an aspect of the disclosure.

### DETAILED DESCRIPTION

The present disclosure relates to a device for assisting the placement of a prosthesis in a corporeal duct, said device being referenced as an ancillary device in the present disclosure. In particular, the disclosure aims to propose an ancillary device configured to assist in the placement of two opposite ends of a prosthesis and junction thereof with a corporeal duct. The disclosure also relates to a kit for anastomosis with no manual suture including said ancillary device.

FIG. 1 illustrates from left to right, a connection assembly 4 (non-visible detail), a prosthesis 3, and the ancillary device 100 of the disclosure in a closed configuration.

The ancillary device 100 is a tool enabling safe handling of the prosthesis 3, as well as facilitating connection thereof to the corporeal duct by means of the connection assembly 4.

The ancillary device 100 includes an elongate body including, on a proximal side, a handling portion 10, and on another distal side a shaping assembly 20 provided with a compression area 21 at a distal end of the device. The body of the device also includes a longitudinal channel, intended to receive the prosthesis 3, and passing through said handling portion and the shaping assembly. In the illustrated aspect, the handling portion 10 is in the form of a hollow tubular enclosure, the hollow interior of its body defining the channel at said handling portion. In particular, the prosthesis is inserted into the channel of the device from a lateral inlet 11 of the handling portion 10 and until positioning one end of the prosthesis 3 at said compression area 21, the latter including a linear channel diameter with a diameter decreasing according to the actuation of the shaping assembly to compress said prosthesis until obtaining a predefined reduced diameter. Alternatively, the prosthesis may be inserted from the compression area as it will be detailed further in the text.

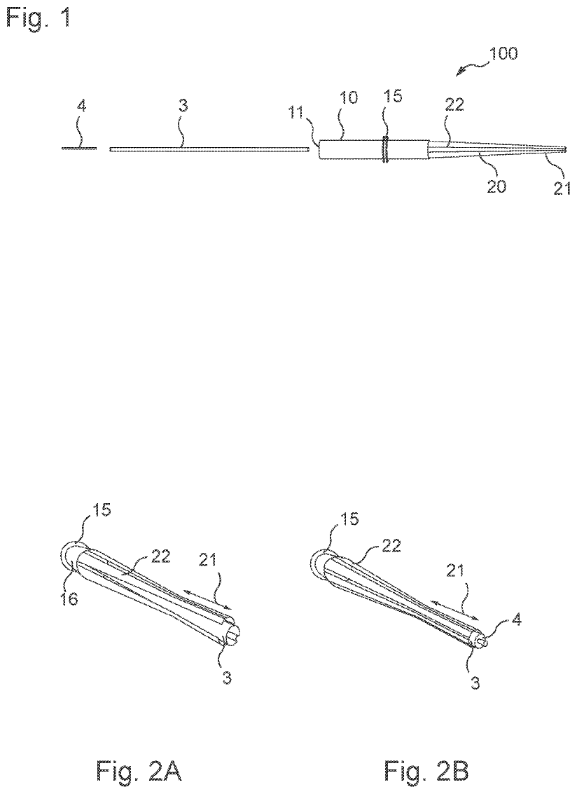

In particular, the shaping assembly 20 allows pinching one end of the prosthesis 3 to compress it radially and facilitate insertion thereof into the corporeal duct 30. To this end, the shaping assembly includes a plurality of lamellae 22 arranged circumferentially around the handling portion 10 and along a longitudinal axis of the device. Said lamellae 22 extend the tubular body of the handling portion 10 and allow defining the through channel at the shaping assembly 20. As illustrated in FIG. 2, the lamellae are arranged at regular intervals, leaving empty spaces between the lamellae.

The shaping assembly 20 includes a released position (FIG. 2A) and a clamped position (FIG. 2B). In the released position, the lamellae 22 define the diameter of the channel 12 allowing easily inserting the prosthesis up to the compression area 21. In the clamped position, the lamellae 22 are tightened, and the diameter of the channel is reduced, in particular at the compression area, so that the internal walls of lamellae compress the prosthesis. Preferably, the diameter of the transverse channel is constant at the compression area 21 and so as to enable a uniform compression of the prosthesis in this area. The diameter of the through channel 12 in the rest of the device is larger than a diameter of the prosthesis, thereby forming a stop preventing the device from being inserted too much into the corporeal duct so that the assembly is inserted without damaging or compressing said body conduit. Hence, the dimensions of the ancillary device 100 are dependent on the dimensions of prostheses to be used, and the same device could be produced with a range of different sizes. In a preferred aspect, the lamellae confer an elongated frustoconical shape on the shaping assembly 20, and include a triangular shape before being planar at the compression area.

According to the illustrated aspect, the ancillary device 100 includes an external ring 15 acting as a means for actuating the clamped position. Indeed, this ring 15 is movable in translation along the device, and allows pushing all lamellae towards the central axis of the channel, in particular when this ring 15 is moved towards or on the lamellae 22 connected to the handling portion. To this end, the handling portion 10 receiving the lamellae, or a lamella base includes a projecting portion on which the ring is fitted to radially compress the lamellae. In the illustrated aspect, this projection 16 is in the form of an external diameter of the handling portion progressively increasing at the handling portion receiving the base of the lamellae. Alternatively, this projection 16 may be integrated at the base of the lamellae 22 (cf. FIG. 8). In both cases, the clamped position is activated by fitting of the ring 15 on this projecting portion 16. Of course, the material of the ancillary device has a certain flexibility enabling this position change. For example, the lamellae are made of titanium or of stainless steel 316L and the handling portion like the ring are made of a biocompatible resin.

In other aspects, other actuating means configured to radially tighten the lamellae may be used, and possibly a manual actuation.

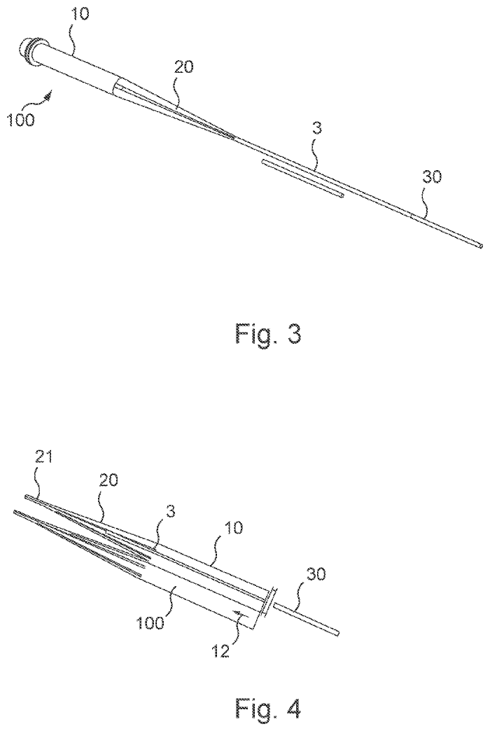

Thus, during the first anastomosis, the practitioner inserts the prosthesis along the through channel, and reduces the diameter of the end of the prosthesis by activating the clamped position of the shaping assembly. Afterwards, the practitioner uses the ancillary device to guide and insert the prosthesis into the so-called top end of the corporeal duct 30. The ancillary device is then removed by pulling it out, for example, towards the free end of the prosthesis (FIG. 3).

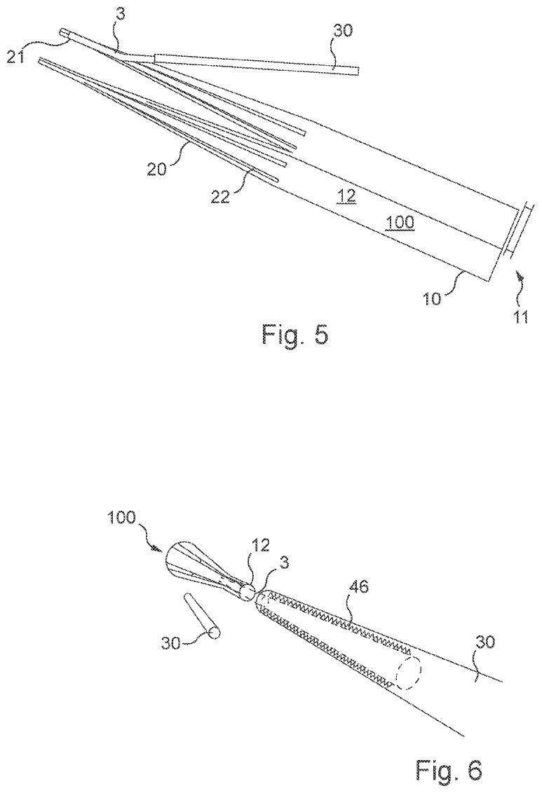

The present disclosure is very advantageous in that it proposes an ancillary device that is optimised to assist in the second anastomosis too. To this end, the disclosure proposes providing the ancillary device 100 with a closable and longitudinal opening giving access to the longitudinal channel 12, and so that a practitioner could easily bring the free end of the prosthesis connected to the corporeal duct up to the compression area 21 of said device (FIG. 4). Hence, this opening allows carefully handling the free end of the prosthesis to position it in said compression area and without the need to make it pass throughout the entire body of the ancillary instrument. In addition, when the length of the prosthesis is limited, it will be possible to position the last millimetres of the prosthesis 3 directly at the compression area 21 and to make the rest of the prosthesis 3 come out through one of the empty spaces between the lamellae 22 (FIG. 5).

The closable opening is very advantageous also in that it facilitates removal of the ancillary device when the two anastomoses have been performed, and that there is no longer any free end towards which this device is pulled out.

In a preferred aspect, the longitudinal opening is configured as an articulated opening splitting the body of the ancillary device into two portions (FIG. 4). This articulation is present along the handling portion and cuts the device into two portions, with opening axes coinciding with the empty spaces between the lamellae.

In one aspect, the actuating ring 15 acts as a closure means allowing holding the closed position when said ring is fitted at the handling portion, and enabling opening when it is disengaged from the body of the device (FIG. 4), or when it is also open into two portions. In another aspect which is not illustrated, the handling portion 20 integrates attachment means in said handling portion. For example, a hook, a clip-on system, etc.

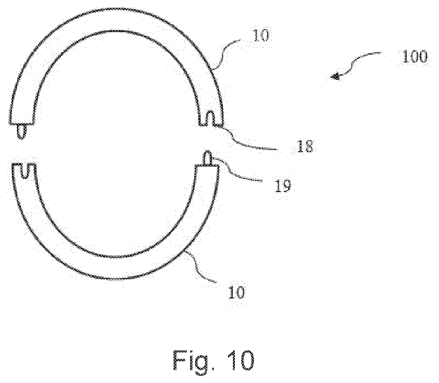

In another aspect which could be combined with any feature of the aspects described hereinbefore, the handling portion of the body of the ancillary device can be opened into two portions that assemble together by means of at least one slide rail in a groove along the body of the handling portion. Hence, opening of the body of the device can be carried out delicately and in a controlled manner to progressively disengage the rail from the groove and expose the prosthesis while holding a support of the latter, and until the ancillary device is completely removed. In the aspect illustrated in FIG. 10, the hollow enclosure of the handling portion 10 is longitudinally divided into two detached portions, and each portion is provided at a longitudinal junction wall between said two portions with a rail 19 and with a groove 18 cooperating with one another.

As illustrated in FIG. 10, the body of the ancillary device is divided into two separate portions at its handling portion 10, which interlock by sliding on top of one another, in particular two hollow semi-cylinders, each semi-cylinder being provided longitudinally at one of its ends with said rail 19, and at the other end with a groove 18 cooperating with the rail 19 and the groove 18 of the other semi-cylinder. Thus, the device is progressively divided into two detached portions. Alternatively, a semi-cylinder may be provided with a pair of rails sliding in a pair of grooves of the other semi-cylinder.

The disclosure also relates to a kit for anastomosis with no manual suture. This anastomosis kit including:
An ancillary device 100 according to the disclosure;
A vascular prosthesis 3 or substitute;
A connection assembly 4 including at least one radial expansion sleeve with spikes 46 and an inflation balloon 40.

As already mentioned, the connection assembly 4 is very advantageous in that it enables connection of the prosthesis 3 with the corporeal duct 30 without the need for manual suturing. As schematically illustrated in FIG. 6, a radial expansion sleeve with spikes 46, also so-called a stent with spikes, is able to perforate the prosthesis 3 and the corporeal duct 30 during radial expansion thereof, which allows securing these elements to one another. The present disclosure proposes a connection assembly 4 designed to cooperate with the ancillary device 100 of the disclosure, and facilitate the anastomosis procedure.

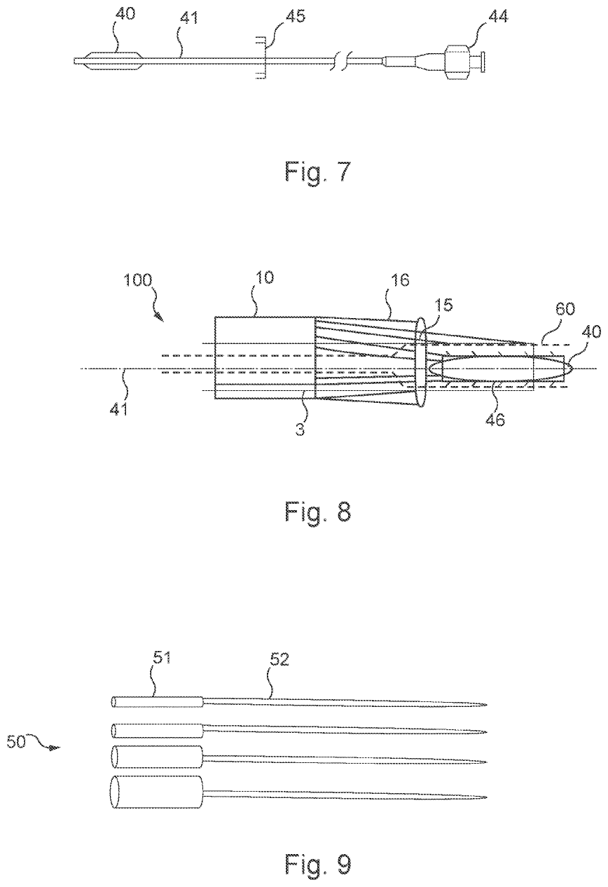

In particular, the radial expansion of the radial expansion sleeve with spikes 46 is obtained by inflation of the inflation balloon inside. In turn, the inflation balloon 40 (FIG. 7) is provided at one end with a catheter 41 connecting said balloon 40 with an inflation syringe 44. Hence, this catheter 41 has a larger length than the length necessary to pass through the channel of the ancillary device 100. In order to accurately insert the balloon 40 and the prosthesis 3, the disclosure proposes providing the connection assembly 4 with an insertion guide system for defining the level of insertion of said connection assembly into the ancillary device, and into the corporeal duct. For example, this guide system is a set of stop elements on the body of the ancillary device and on the connection assembly, configured to limit the depth of insertion of the latter.

In an aspect schematically illustrated in FIG. 7, the catheter 41 of the inflation balloon 40 is provided with a stop element 45 of the guide system in the form of the fins including peripheral hooks configured to hook at the inlet 11 of the ancillary device or at one end of the prosthesis. Hence, the connection assembly 4 is inserted into the channel of the ancillary device until stoppage thereof by anchoring of the hooks of the fins 45 of the catheter 41 to the ancillary device. For example, the fins 45 have at least two branches, and preferably four or six branches that fit onto the hollow cylindrical base of the ancillary.

In another aspect, the fins 45 define stops configured to fit into the channel of the ancillary device 100 and be stopped at the inner stops of the ancillary device or by a wall of the prosthesis. In a preferred aspect, the fins 45 are movable in translation along the catheter 41. Thus, a practitioner can limit, on a case-by-case basis, the insertion depth of the connection assembly, which is very advantageous during the second anastomosis. Indeed, during the second anastomosis, positioning the stent and the prosthesis should be carried out under visual control, and that being so in a case-by-case basis. Once this desired insertion level is determined, the practitioner could change the position of the fins so that they abut against the prosthesis and the connection assembly is immobilised.

In an aspect illustrated in FIG. 8, the kit also comprises an enclosure 60 for protecting said radial expansion sleeve with spikes 46 allowing preserving the integrity of the spikes until insertion thereof into the corporeal duct, and in particular during the step of compressing the prosthesis 3. This enclosure is arranged around the stent with spikes 46, said stent with spikes being inserted into the prosthesis mounted on the balloon 40 and surrounded by said protective enclosure. In particular, a diameter of the enclosure surrounding the balloon is slightly smaller than the diameter of the inner orifice at the compression area 21, said enclosure is therefore not compressed, but only the end of the prosthesis to be inserted into the corporeal duct 30 without being able to compress the stent with spikes 46 protected by the enclosure 60.

The protective enclosure 60 is also retained during the step of inserting the prosthesis and the connection assembly 4 into the corporeal duct 30, to then be removed before the step of inflating the balloon 40. Advantageously, the disclosure proposes providing said enclosure 60 with a peelable or frangible system allowing opening said enclosure by pulling from its proximal end extending towards the outside of the corporeal duct. In FIG. 8, the dotted lines indicate peelable or frangible axes allowing dividing the enclosure into two portions to facilitate removal thereof. Preferably, and in an aspect not illustrated, a proximal end of the enclosure 60 is split at four levels to facilitate peeling of the enclosure for removal thereof. Advantageously, this aspect also allows facilitating the passage of the fins.

Finally, the kit of the disclosure also includes a diameter tester 50 including a set of tubular bodies 51, such as hollow cylinders 51 having different diameters, each cylinder 51 including a grip 52 by which the practitioner handles the cylinder in order to insert it into the corporeal duct (FIG. 9). In particular, the practitioner tests the different cylinders until finding the cylinder diameter that could be inserted into the corporeal duct with limited clearance in said duct. Hence, the diameters of the cylinders 51 are slightly smaller than the inner diameters of corporeal ducts for which the anastomosis is intended. These cylinders may also allow carrying out a complementary external compression of the prosthesis when this proves necessary.

Thus, the disclosure proposes an anastomosis kit allowing perfectly positioning a prosthesis in a corporeal duct, said prosthesis associated with a stent with spikes to ensure the junction of said prosthesis with no manual suture. In particular, the device of the disclosure is very advantageous in that it allows assisting during junction of two ends of the prosthesis thanks to its characteristic closable opening.

What is claimed is:

1. An ancillary device for anastomosis, said device including an elongate body provided with a longitudinal and through channel configured to receive a tubular prosthesis therein, the body of the device including, on a proximal side, a handling portion formed by a hollow enclosure, preferably tubular shaped and, on a distal side, a shaping assembly formed by a plurality of the lamellae defining a compression area of said prosthesis, characterised in that:
the body of the device includes a closable longitudinal opening giving access to the longitudinal channel at said handling portion and of the shaping assembly.

2. The device according to claim 1, wherein the longitudinal opening is configured as an opening splitting said body into two portions, and wherein the two portions are assembled together by means of at least one rail and a groove cooperating with one another and being respectively provided on each of the two portions along the body of the handling portion.

3. The device according to claim 1, wherein the device includes an open configuration, and a closed configuration controllable by engagement and disengagement of the rail in the groove, and preferably means for locking said closed configuration.

4. The device according to claim 1, the lamellae of the shaping assembly are arranged circumferentially and according to predefined intervals around the handling portion and according to an axis of the device, and in which device the lamellae have a released position and a clamped position defining respectively an initial diameter and a reduced diameter of the channel receiving the prosthesis at said compression area.

5. The device according to claim 4, the device including an actuating means configured to radially compress the lamellae and activate their clamped position.

6. The device according to claim 1, wherein the handling portion includes one or more projecting portions at a junction area with the lamellae of the shaping assembly, said one or more projecting portions being configured for compressing the lamellae radially when it is pushed towards the inside of the device.

7. The device according to claim 6, including an external ring movable in translation along the body of the device, said ring being able to fit onto said one or more projecting portions to activate the clamped position of the lamellae.

8. An anastomosis kit including:
an ancillary device according to claim 1;
a prosthesis or vascular substitute
a connection assembly including at least one radial expansion sleeve with spikes and an inflation balloon.

9. The anastomosis kit according to claim 8, said kit including an insertion guide system including a set of stop elements on the body of the ancillary device and on the connection assembly, configured to limit the depth of insertion of the connection assembly into the channel of the ancillary device.

10. The anastomosis kit according to claim 8, wherein the inflation balloon includes a catheter for connecting said balloon to an inflation syringe, and said catheter includes one or more fins configured to anchor to the body of the ancillary device or to the prosthesis, and to limit a depth of insertion of the connection assembly into the channel of the ancillary device.

11. The anastomosis kit according to claim 10, wherein said one or more fins include peripheral hooks configured to hook at a proximal end of the handling portion of the ancillary device or at one end of the prosthesis.

12. The anastomosis kit according to claim 10, wherein said one or more fins are movable in translation along the catheter and include a locked configuration and an unlocked configuration, respectively limiting or enabling mobility of said one or more fins.

13. The anastomosis kit according to claim 8, including a peelable or frangible protective enclosure surrounding the radial expansion sleeve with spikes.

14. The anastomosis kit according to claim 13, wherein the enclosure has at least one peelable or frangible longitudinal axis.

15. The anastomosis kit according to claim 8, including a diameter tester including at least one tubular body with a predefined diameter connected to a grip, and preferably a plurality of the tubular bodies respectively connected to a grip.
